(19) United States
(12) Patent Application Publication
(10) Pub. No.: US 2009/0216340 A1
(43) Pub. Date: Aug. 27, 2009

(54) Articulation Joint Which Can Be Locked By Means of Elastic Deformation and Knee Prosthesis Using Same

(76) Inventor: Pierre Martin, Loriol (FR)

Correspondence Address: CLARK & BRODY, 1090 VERMONT AVENUE, NW, SUITE 250, WASHINGTON, DC, 20005, US

(21) Appl. No.: 11/992,770

(22) PCT Filed: Sep. 22, 2006
(86) PCT No.: PCT/EP2006/066651

§ 371 Date: Apr. 17, 2009

(30) Foreign Application Priority Data
Sep. 30, 2005 (FR) FR0510014

(51) Int. Cl. A61F 2/64; F16C 11/06

(52) U.S. Cl. 623/43; 403/102

(57) ABSTRACT

The invention relates to an articulation joint which can move in a controlled manner between two elements that can rotate in relation to one another about an axis Δ, comprising: a hub (6) which is equipped with a cylindrical bore (7) having a Δ axis and which is intended to be fixed to the first element; and a shaft (10) which is engaged in the bore in the hub (6), such that it can move in relation to the hub in rotation about axis Δ, and which is intended to be fixed to the second element. The invention is characterised in that the hub (6) and the shaft (10) are elastic and can deform under a stress (F3, F4) which is perpendicular to the Δ axis and which is greater than a given value, in order to lock or brake the Δ axis rotation.

The present invention relates to the technical field of devices used for making articulation with controlled movements between two elements mobile in rotation relative to one another, according to a Δ axis.

In a preferred though non-exclusive application the invention relates to controlled movement articulations utilised in leg prostheses to form an artificial knee ensuring articulation between an adaptation element on a thigh stump and an element, known as tibial, having an artificial foot at its distal end.

The articulation or artificial knee of such a prosthesis must then be able to ensure two functions corresponding to different phases of walking for the user of the prosthesis.

First, the controlled movement articulation must be able to be blocked in the phases, known as walking support, during which the weight of the body of the user bears on the prosthesis. Next, the articulation must be able to be unblocked to enable pendular movement of the tibial segment during a walking phase during which the user of the prosthesis has transferred his weight to his valid leg.

To ensure this double function, the controlled movement articulation must comprise blocking and unblocking means, allowing passage for these two operating modes of free rotation and locked rotation.

For this purpose a French patent FR 2 261 742 has proposed using an articulation comprising a pivot and a controlled pneumatic jack for blocking or allowing pendular movements of the tibial segment.

With respect to its functions for blocking the rotational movement and unblocking the latter such an articulation is satisfactory, though it does have the drawback of making components relatively heavy and, especially, relatively complex and bulky control systems of the pneumatic jack, such that the prosthesis ensemble has both a relatively significant weight and seemingly prohibitive operating costs. With respect to weight, it must also be noted that its substantial character makes such a prosthesis difficult to use by the aged.

So as to simplify the locking mechanism, it has been proposed especially in documents DE 1030508, DE 841193 and FR 1099570 to employ an articulation whereof either the axis or the housing is elastic to enable, under the effect of a constraint, relative translation of the two mobile elements and to place in contact pieces or friction surfaces which brake rotation. Thus, when at rest and in the absence of constraint, the friction surfaces are distanced from one another, whereas under the effect of the constraint they are in contact.

Such an embodiment especially has the disadvantage of requiring friction surfaces or friction pieces which add weight to the articulation. This embodiment also has the disadvantage of not always ensuring efficacious locking of the articulation. Thus, there is a need for a novel articulation which, while offering possibilities of locking and blocking of its rotational movement, has a simplified design which limits or even eliminates those accessories and devices necessary for its blocking, such that it is possible to reduce the weight, both in terms of the articulation per se as in the control means necessary for ensuring its temporary blocking, while guaranteeing the efficacy of locking.

To meet these objectives, the invention relates to a controlled movement articulation between two elements mobile in rotation relative to one another about a Δ axis, comprising:
a hub which is provided with a cylindrical bore of Δ axis and which is intended to be fixed to the first element,
and a shaft which is engaged in the bore of the hub to be mobile relative to the Δ axis. This shaft is intended to be made solid with the second element.

According to the invention, the articulation is characterised in that the hub and the shaft are elastically deformable so as to deform under the effect of a constraint perpendicular to the Δ axis and greater than a given value for ensuring locking or geometric braking of the Δ axis rotation.

Therefore, according to a characteristic of the invention, the internal surface of the bore and the external surface of the shaft are in contact and have a complementary form and the hub and the shaft are elastically deformable such that:
at rest, the internal surface of the bore and the external surface of the shaft have a cylindrical revolution form of Δ axis which enables rotation about the Δ axis,
under the effect of a constraint perpendicular to the Δ axis, greater than a given value, the internal surface of the bore and the external surface of the shaft deform conjointly, this reversible joint deformation locking or braking the Δ axis rotation.

According to the invention, the joint deformation of the hub and of the shaft causing locking of the rotational movement about the Δ axis can take on different forms to the extent where it is reversible, such that when the constraint ceases the hub and the shaft regain their original conformation enabling rotation about the Δ axis. The joint deformation must allow reversible passage of the surfaces in contact or guidance of the hub and of the shaft, from a form of revolution enabling rotation, to a form which is no longer revolution and causes braking or geometric locking of the rotation.

According to a characteristic of the invention, the hub and the shaft are made such that the internal surface of the cylindrical bore and the external surface of the shaft form an oval by retaining a complementary form under the effect of the constraint perpendicular to the Δ axis.

Similarly, the hub and the shaft can be made differently to the extent where the internal surface of the bore of the hub and the external surface of the shaft are likely to know reversible joint deformation for locking of the rotation about the Δ axis, and to the extent where there is complementarity of the forms of the internal surface of the hub and external surface of the axis along of the functioning of the articulation.

According to a preferred though non-exclusive embodiment of an articulation according to the invention, the hub comprises a cylindrical sleeve provided at the level of its external surface with fixing means for the first element, whereas the shaft comprises a cylindrical sleeve which is concentric and adjusted to the interior of the sleeve of the hub. This hollow shaft comprises fixing means of the second element at the level of its internal surface.

According to the invention, the cylindrical sleeves can be made of any material having the requisite mechanical characteristics.

According to a characteristic of the invention, the internal surface of the sleeve of the hub and/or the external surface of the sleeve of the shaft are covered or constituted by material having low friction coefficient.

According to the invention, it can also be envisaged to interpose a ring made of material with low friction coefficient between the hub and the shaft.

According to another characteristic of the invention, the articulation comprises stop means prohibiting relative translation of the hub and of the shaft according to the Δ axis.

The invention also relates to a leg or artificial leg prosthesis comprising at least one adaptation element on a thigh stump, an artificial tibial element and linking means articulated between the adaptation element and the tibial segment. The articulated linking means comprise at least one articulation according to the invention connecting the adaptation element and the tibial element.

Of course, the different characteristics of the invention mentioned above can be used with one another according to different combinations when they are not exclusive to each other.

Also, various other characteristics of the invention will emerge from the following description in reference to the attached diagrams which show by way of non-limiting examples embodiments of the object of the invention.

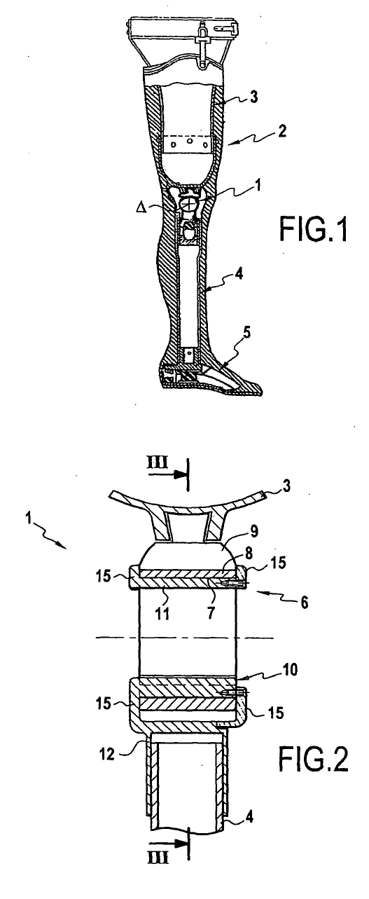

FIG. 1 is a partially exploded elevation, in a side view of a total leg prosthesis according to the invention.

FIG. 2 is an axial section of the controlled movement articulation according to the invention used on the leg prosthesis, such as illustrated in FIG. 1.

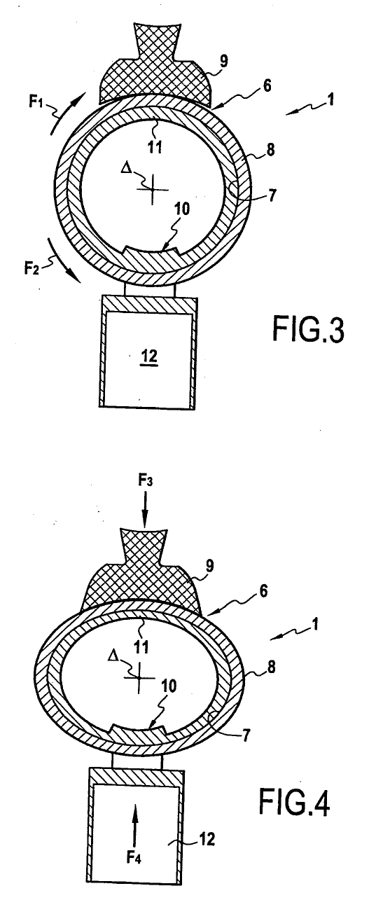

FIG. 3 is a transversal section, according to the line III-III of FIG. 2, showing the controlled movement articulation according to the invention in a non-deformed state enabling free rotation of the articulation.

FIG. 4 is a transversal section, similar to FIG. 3, showing the articulation according to the invention in a deformed state under load, locking all rotation of the articulation according to the Δ axis.

A controlled movement articulation, such as illustrated in FIGS. 1 and 2 and designated in its entirety by reference numeral 1, is, in a preferred application, intended to ensure articulation of a leg prosthesis 2, such as illustrated schematically in FIG. 1. Such a leg prosthesis 2 first comprises an adaptation element 3, intended to be fixed on the stump, not shown, of the thigh of a user. The prosthesis 2 also comprises a tibial segment 4, connected to the adaptation element 3 by the articulation 1 and provided opposite the articulation 1 with an artificial foot 5. The adaptation element 3, the tibial segment 4 and the artificial foot 5 can be made in any appropriate manner known to the expert. Also, the embodiment of the adaptation element 3 of the tibial segment 4 and of the artificial foot 5 does not fit within the scope of the present invention which relates more particularly to the controlled movement articulation 1, such that it is not necessary to describe it further here.

The articulation 1 is intended to form an artificial knee enabling relative rotation about a Δ axis of the adaptation element 3 and of the element tibial 4. Also, the controlled articulation 1 is designed to allow blocking of rotation about the Δ axis, so as to permit support or transfer of the weight of the user of the prosthesis 2 to the foot 5.

To allow rotational lockable or controllable movement about the Δ axis between the adaptation element 3 and the tibial element 4 the controlled movement articulation 1 first comprises a hub 6 which is provided with a cylindrical bore 7 of Δ axis. The hub 6 is intended to be fixed to the adaptation element 3. According to the example illustrated the hub 6 comprises a tubular sleeve 8 which has, at rest and without external stress, at least at the level of its bore or internal wall 7 a cylindrical revolution form of a Δ axis. The cylindrical sleeve 8 is equipped at the level of its external surface with fixing means 9 on the adaptation element 3.

The articulation 1 then comprises a shaft 10 which engages in the bore 7 of the hub 6 to be mobile relative to the hub in Δ axis rotation. The shaft 10 is intended to be fixed to the tibial element 4. According to the example illustrated, the shaft 10 comprises a tubular sleeve 11 which has, at rest and without external stress, at least at the level of its external wall a cylindrical revolution form of Δ axis. As is evident more particularly from FIG. 2, the tubular sleeve 11 has a form complementary to that of the tubular sleeve 8 such that the external wall of the sleeve 11 is in contact with the internal wall of the sleeve 8 defining the bore 7. The tubular sleeve 11 finally comprises fixing means 12 on the tibial element 4, the fixing means 12 being connected to the sleeve 11 at the level of its internal surface.

The cylindrical sleeves 8 and 11 are made from material enabling reversible elastic deformation of the sleeves, as will be apparent hereinbelow.

The resulting articulation 1 according to the invention functions as follows.

When any constraint perpendicular to the Δ axis is applied to the articulation 1 or when the constraints perpendicular to the Δ axis are insufficient to cause elastic deformation of the sleeves 8 and 11, the guide or contact surfaces between the sleeves 8 and 11 have a cylindrical revolution form of Δ axis which enables rotation about this Δ axis in one direction or another, as indicated by arrows F1 or F2. This state of rest corresponds, in the case of the leg prosthesis 2, to moment where the weight of the user is transferred to the valid leg, the tibial part of the hip prosthesis then being able to describe pendular movement. Of course, it could be feasible to use with the articulation 1 stop means, not shown, for defining the possible amplitude in rotation of the movement of the tibial segment 4 relative to the adaptation element 3. Similarly, to facilitate rotation movements between the two sleeves, one or the other of their surfaces in contact could be covered by material with low friction coefficient, such as for example a Teflon®-based material. It could also be feasible to interpose between the hub and the shaft a ring of slight thickness made of Teflon® or other material with low friction coefficient.

However, when a constraint such as indicated by the arrows F3, F4 is applied perpendicularly to the Δ axis, with sufficient intensity to deform the sleeves 8 and 11, their surfaces in contact lose their cylindrical revolution form, according to the example illustrated, to form an oval as shown in FIG. 4. This then produces locking or geometric braking as opposed to braking or locking resulting from friction alone. In this way all rotational movement about the Δ axis is blocked or braked. This situation corresponds, in the case of the leg prosthesis 2, to the moment where the user bears his weight on the prosthesis.

It appears that the articulation 1 according to the invention produces simple locking or braking of the rotation, and this with a minimum of components such that the articulation according to the invention is light and not bulky.

Also, according to the example illustrated, the articulation 1 comprises stop means 15 preventing all relative translation, according to the Δ axis, of the hub and of the shaft.

It is understood that various modifications can be made to the invention without departing from its scope.

1. Articulation with controlled movements between two elements (3 and 4) mobile in rotation relative to one another about a Δ axis, comprising:
a hub (6) which is provided with a cylindrical bore (7) of Δ axis and which is intended to be fixed to the first element (3),
and a shaft (10) which is engage in the bore of the hub (6) to be mobile relative to the hub in Δ axis rotation and which is intended to be fixed to the second element (4),
characterised in that the hub (6) and the shaft (10) are elastic such that they can deform under the effect of a constraint (F3, F4) perpendicular to the Δ axis, greater than a given value, to ensure locking or geometric braking of the Δ axis rotation.

2. Articulation as claimed in claim 1 characterised in that the internal surface of the bore (7) and the external surface of the shaft (10) are in contact and have a complementary form, and in that the hub (6) and the shaft (10) are elastically deformable such that:
at rest, the internal surface of the bore (7) and the external surface of the shaft (10) have a cylindrical revolution form of Δ axis which enables rotation about the Δ axis,
under the effect of a constraint (F3, F4) perpendicular to the Δ axis, greater than a given value, the internal surface of the bore (7) and the external surface of the shaft (10) deform conjointly, this reversible conjoint deformation locking or braking the Δ axis rotation.

3. Articulation as claimed in claim 1, characterised in that the hub (6) and shaft (10) are made such that the internal surface of the cylindrical bore (7) and the external surface of the shaft form an oval by preserving a complementary form under the effect of the constraint perpendicular to the Δ axis.

4. Articulation as claimed in claim 1 characterised in that:
the hub (6) comprises a cylindrical sleeve (8) provided at the level of its external surface with fixing means (9) for the first element (3),
the shaft (10) comprises a cylindrical sleeve (11) which has a form complementary to that of the sleeve (8) of the hub and which comprises at the level of its internal surface fixing means (12) for the second element.

5. Articulation as claimed in claim 4, characterised in that the internal surface of the sleeve (8) of the hub and the external surface of the sleeve (11) of the shaft are covered with or constituted by a material with low friction coefficient.

6. Articulation as claimed in claim 4, characterised in that it comprises a ring with low friction coefficient interposed between the hub and the shaft.

7. Articulation as claimed in claim 1, characterised in that it comprises stop means (15) prohibiting any relative translation according to the Δ axis between the hub and the shaft.

8. A knee prosthesis comprising at least one adaptation element (3) on a thigh stump, an artificial tibial element (4) and linking means (1) articulated between the adaptation element and the tibial segment, characterised in that the linking means comprise at least one articulation as claimed in claim 1.
